(19) United States
(12) Patent Application Publication
(10) Pub. No.: US 2006/0276688 A1
(43) Pub. Date: Dec. 7, 2006

(54) Hemostasis flushing device

(75) Inventor: Vihar C. Surti, Winston-Salem, NC (US)

Correspondence Address: BRINKS HOFER GILSON & LIONE/CHICAGO/COOK, PO BOX 10395, CHICAGO, IL, 60610, US

(73) Assignee: Wilson-Cook Medical Inc., Winston-Salem, NC (US)

(21) Appl. No.: 11/435,192

(22) Filed: May 16, 2006

Related U.S. Application Data
(60) Provisional application No. 60/681,648, filed on May 17, 2005.

(51) Int. Cl. A61B 1/00

(52) U.S. Cl. 600/154

(57) ABSTRACT

A hemostasis flushing device includes a main body having a proximal end and a distal end wherein a lumen extends between the proximal end and the distal end of the main body. A flush port is disposed between the proximal end and the distal end of the main body for passing fluid into or out of the main body. An access port is disposed along the proximal end of the main body for receiving an inserted elongate medical device. An attachment port is disposed on the distal end of the main body for allowing a hemostasis flushing device to be connected to an endoscope. The attachment port may also include an annular tip, wherein the annular tip provides an opening for the elongate medical device to extend into the accessory channel of the endoscope.

### RELATED APPLICATIONS

This application claims priority to provisional application No. 60/681,648 filed on May 17, 2005, the entire disclosure of which is incorporated by reference herein.

### FIELD OF THE INVENTION

The present invention relates to the field of medical devices, and more particularly to medical devices utilizing endoscopic tools for facilitating hemostasis in a gastrointestinal passageway.

### BACKGROUND

Conventional medical devices are introduced into a gastrointestinal passageway through an endoscope for providing hemostasis during medical procedures. A number of medical devices, such as clips, snares and needles, are inserted into an accessory channel of an endoscope to prevent bleeding during these procedures. However, the amount of bleeding is difficult to contain due to several factors, such as the size of the medical device, the patient's coagulation status, and the location of the gastrointestinal passageway of the ongoing medical procedure.

Often times, excessive bleeding in the gastrointestinal passageway of the patient makes it difficult to visualize or access the particular gastrointestinal passageway after inserting the medical device directly into the accessory channel of the endoscope. Additionally, many endoscopes fail to provide visual access to a target anatomy during bleeding without the use of additional procedures, such as fluoroscopy. Nonetheless, there is increased difficulty in visualizing the gastrointestinal passageway while maneuvering the medical device through the accessory channel of the endoscope due to bleeding at the connection point between the medical device and the accessory channel of the endoscope. For example, when a medical device, such as a bi-polar probe, needle, and other elongated device, is passed through an accessory channel of an endoscope the medical device often becomes improperly flushed around the accessory channel from the excessive bleeding. These flushing problems can result in leaking around the accessory channel of the endoscope and the medical device. In addition to leaking, flushing problems around the accessory channel of the endoscope can result in blockage from the accumulation of fluids, such as blood, and lead to other complications. Both blockage and leaking problems can cause decreased pressure in the corresponding accessory channel of the endoscope and result in flushing fluid being delivered to a particular site at a slower rate during time-sensitive medical procedures.

Due to the complexity of medical procedures involving medical devices, physicians often need to repeatedly insert and remove the particular medical device at different stages of surgery to permit the delivery of flushing fluid to the target site of the gastrointestinal passageway. For instance, current procedures may require a physician to insert a medical device, such as an injectable catheter, into an accessory channel of an endoscope during the beginning of a surgical procedure to deliver medicine to a patient and then subsequently remove the medical device. During the same procedure, the physician may insert a different medical device, such as a syringe, into the accessory channel of the endoscope to deliver fluids to the patient for flushing the target site of the gastrointestinal passageway. After flushing the target site, the physician must remove the syringe and then reinsert the initial medical device being utilized for performing the ongoing medical procedure. The time required to exchange the medical device adds significant time to the ongoing medical procedure. Additionally, it remains difficult to flush the area surrounding the accessory channel of the endoscope as coagulated blood continues to build up between the accessory channel of the endoscope and the medical device.

It is desirable to provide a hemostasis flushing device that allows a plurality of medical devices to be easily inserted and removed from an endoscope during medical procedures to reduce excessive bleeding. Also, there exists a need for a hemostasis flushing device that provides for flushing within a lumen of the device, and provides for flushing around an accessory channel of an attached endoscope, while providing access and visual clarity of a target site during medical procedures involving the endoscope.

### SUMMARY

In one aspect of the invention, a hemostasis flushing device comprises a main body having a proximal end and a distal end wherein a lumen extends between the proximal end and the distal end of the main body. The hemostasis flushing device further comprises a flush port disposed between the proximal end and the distal end of the main body for passing fluid through the lumen of the main body. The hemostasis flushing device also comprises an access port disposed along a proximal end of the main body for receiving an elongate medical device extending through the lumen of the main body. An attachment port is disposed along the distal end of the main body wherein the attachment port is connectable to an endoscope for receiving the elongate medical device extending through the lumen of the main body. The multiple ports, such as the flush port and access port, of the hemostasis flushing device provides for flushing inside the lumen of the hemostasis flushing device while also providing for flushing around the elongate medical device and an accessory channel of the attached endoscope during medical procedures for facilitating hemostasis.

In one aspect of the present invention, the attachment port further comprises an annular tip for receiving the elongate medical device extending through the lumen of the main body. The hemostasis flushing device may also include an infusion device attached to the flush port for flushing fluid through the lumen of the main body. The flush port is in fluid communication with the lumen of the main body and is configured to permit ingress and egress of fluid from the lumen of the main body. The flush port also provides a seal for sealing the flush port. The hemostasis flushing device may include a varying diameter wherein the proximal end of the main body includes a larger diameter and the distal end of the main body includes a smaller diameter for facilitating fluid flow within the hemostasis flushing device.

The hemostasis flushing device may be engaged to the endoscope by utilizing a metal insert having a lip for connecting the endoscope to the attachment port of the hemostasis flushing device to the endoscope. The types of elongate medical devices that may be inserted into the lumen of the main body include a needle, probe, clip, syringe or similar elongate medical device. Multiple elongate medical devices may be inserted into a port of the hemostasis flushing device for facilitating hemostasis in a blood vessel. For example, a syringe may be inserted into the flush port while a probe and a needle are inserted through the access port of the hemostasis flushing device. The elongate medical device may also include a hydrophilic coating for navigating the elongate medical device through the lumen of the main body.

In another aspect of the present invention, a method of flushing around an elongate medical device comprises the step of providing a hemostasis flushing device comprising a main body having a proximal end and a distal end wherein a lumen extends between the proximal end and the distal end of the main body for inserting an elongate medical device. The method also comprises the steps of providing a flush port disposed between the proximal end and the distal end of the main body for passing fluid into the lumen of the main body, and providing an access port disposed along a proximal end of the main body for receiving the elongate medical device extending through the lumen of the main body. The method comprises the additional step of providing an attachment port disposed along the distal end of the main body wherein the attachment port is connectable to an endoscope for receiving the elongate medical device extending through the lumen of the main body. Additionally, the method comprises the steps of attaching the attachment port to an endoscope and inserting an elongate medical device through the lumen of the main body. The method also comprises the steps of inserting an infusion device to the flush port of the hemostasis flushing device and flushing fluid through the flush port into the lumen of the main body to remove bodily fluid from around the infusion device.

### BRIEF DESCRIPTION OF THE DRAWINGS

Several embodiments of the present invention will now be described by way of example with reference to the accompanying drawings, in which:

FIG. 1 shows a side view of a hemostasis flushing device of the present invention;

FIG. 2 shows a cross-sectional view of the hemostasis flushing device of FIG. 1 of the present invention;

FIG. 3 shows a partial cross-sectional view of the hemostasis flushing device of FIG. 1 having an elongate medical device disposed in a lumen of the hemostasis flushing device of the present invention;

FIG. 4 shows a partially sectioned end view of annular tip of the hemostasis flushing device of FIG. 3 wherein the elongate medical device extends into an accessory channel of an endoscope of the present invention;

FIG. 5 shows a partially sectioned end view of an access port of the hemostasis flushing device of FIG. 3 wherein the elongate medical device extends into a lumen of the hemostasis flushing device of the present invention;

FIG. 6 shows a perspective view of an endoscope connectable to an attachment port of the hemostasis flushing device of the present invention;

FIG. 7 shows a perspective view of a hemostasis flushing device engaged to an accessory channel of the endoscope of FIG. 6 of the present invention;

FIG. 8 shows a partial cross-sectional view of the hemostasis flushing device engaged to the accessory channel of the endoscope of FIG. 7 of the present invention; and

FIG. 9 shows a partial cross-sectional view of the hemostasis flushing device frictionally engaged to the accessory channel of the endoscope of FIG. 8 of the present invention.

### DESCRIPTION OF THE INVENTION

The invention is described with reference to the drawings in which like elements are referred to by like numerals. The relationship and functioning of the various elements of this invention are better understood by the following detailed description. However, the embodiments of this invention are not limited to the embodiments illustrated in the drawings. It should be understood that the drawings are not to scale and in certain instances details have been omitted, which are not necessary for an understanding of the present invention, such as conventional fabrication and assembly.

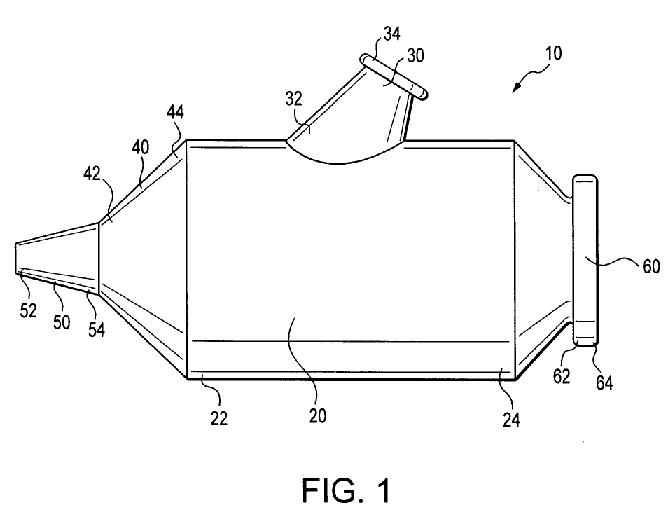

Referring now to FIGS. 1-5, an embodiment of a hemostasis flushing device 10 of the present invention is shown. For ease of reference but without limiting the scope of the claims, the present invention will be described in connection with an endoscope 200 of the type shown in FIG. 6.

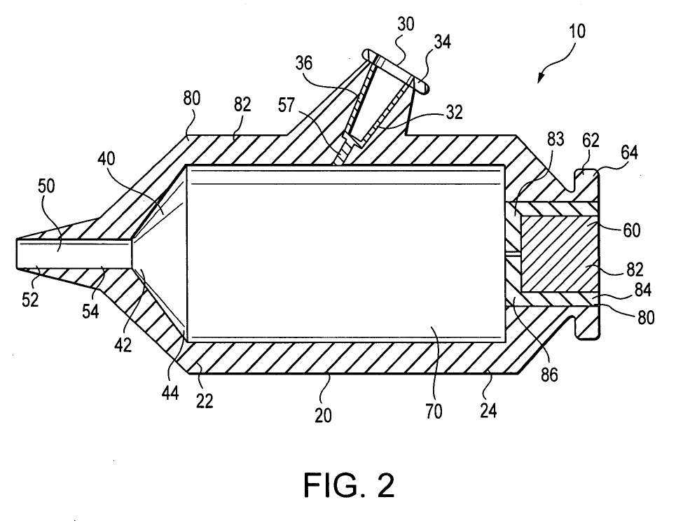

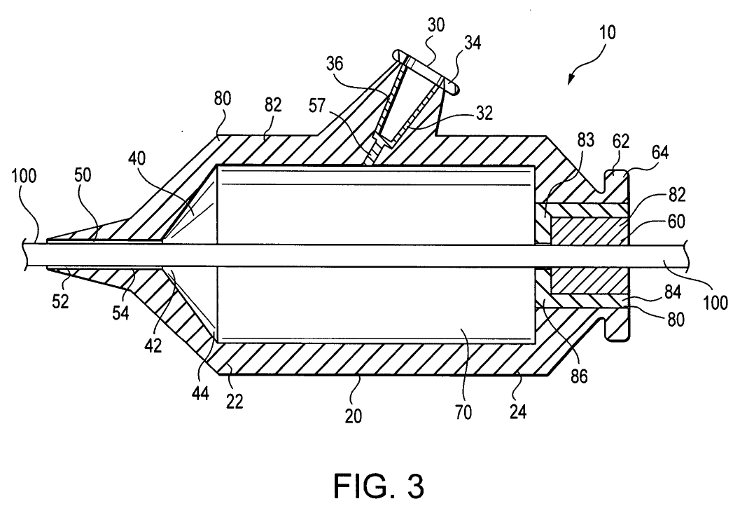

In the embodiment of the present invention illustrated in FIGS. 1-5, the hemostasis flushing device 10 includes a main body 20, an access port 60, an attachment port 40 and a flush port 30. The main body 20 of the hemostasis flushing device 10 comprises a proximal end 24 and a distal end 22 wherein a lumen 70 (FIG. 2) extends between the proximal end 24 and the distal end 22 of the main body 20. The access port 60 is disposed along the proximal end 24 of the main body 20 for receiving an elongate medical device 100 inserted into the access port 60 and extended through the lumen 70 of the main body 20 (see FIG. 3). The attachment port 40 is disposed along the distal end 22 of the main body 20 and further comprises an annular tip 50. The attachment port 40 is frictionally engaged to the opening of the accessory channel 204 of the endoscope 200, wherein the annular tip 50 is received and secured in the opening of the accessory channel 204 of the endoscope 200 (see FIG. 6). The elongate medical device 100 (see FIG. 3) extends through the access port 60 through the lumen 70, and through the annular tip 50 as it passes into the endoscope 200. The flush port 30 is disposed between the proximal end 24 and distal end 22 of the main body 20 and is configured for receiving fluids for flushing around the elongate medical device 100 as it extends through the lumen 70 of the main body 20.

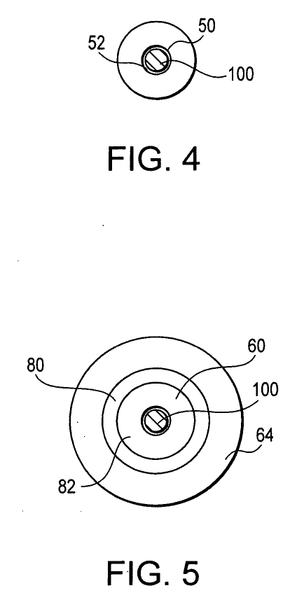

Referring to FIG. 2, the lumen 70 of the hemostasis flushing device 10 receives various medical devices, such as a probe, needle, snare, clipping device, and other similar elongate devices. The lumen 70 of the hemostasis flushing device 10 allows the elongate medical device 100 to be freely inserted and removed during medical procedures (FIG. 3). The lumen 70 extends from the access port 60 of the hemostasis flushing device 10 to the annular tip 50 of the hemostasis flushing device 10 (FIG. 4). Upon insertion into the lumen 70 of the main body 20, the elongate medical device 100 can be maneuvered longitudinally and rotationally along the lumen 70 of the main body 20 into an accessory channel 204 of the endoscope 200 (FIG. 6) for purposes of introduction to a target site of the patient. The lumen 70 also provides a reservoir for fluid passing out of the accessory channel 204 of the endoscope 200. Fluid in the lumen 70 is accumulated until it is removed through the flush port 30, or the fluid is removed through the annular tip 50 by detaching the entire hemostasis flushing device 10 from the accessory channel 204 of the endoscope 200.

In the embodiment illustrated in FIGS. 1-3, the hemostasis flushing device 10 is elongate and has a cylindrical shape. However, the shape of hemostasis flushing device 10 can include other shapes, such as rectangular, elliptical, or any combination thereof. The optimal length of the hemostasis flushing device 10 is determined by considering factors such as design and material used, as well by what is determined through experimentation to work best. The hemostasis flushing device 10 may be preferably formed of molded plastic material or metal which may be repeated sterilized by medical providers during or between medical procedures. Alternatively, the hemostasis flushing device 10 may be initially sterilized and then disposed of.

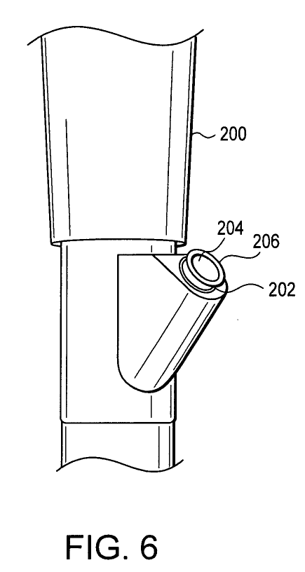

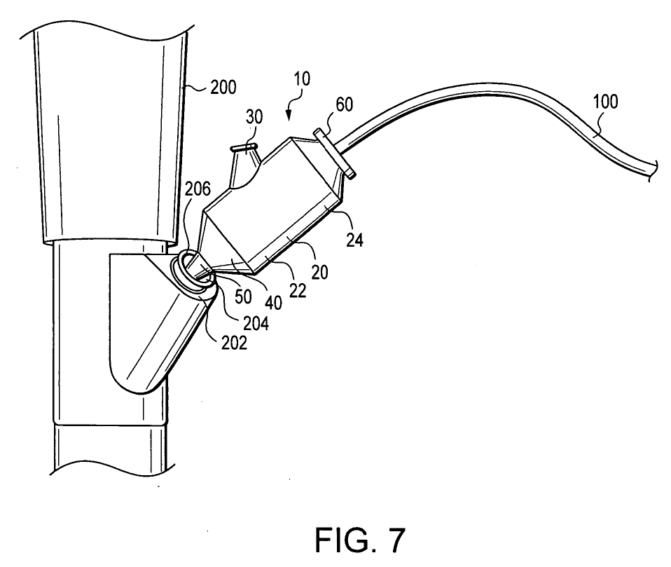

As shown in FIG. 3, the access port 60 of the hemostasis flushing device 10 includes a proximal end 64 and a distal end 62. The distal end 62 of the access port 60 is disposed along the proximal end 24 of the main body 20 and the proximal end 64 of the access port 60 extends away from the proximal end 24 of the main body 20. The access port 60 provides a passageway for receiving the elongate medical device 100 as the device 100 extends into and through the lumen 70 of the main body 20 (FIG. 5) and exits from the annular tip 50 of the hemostasis flushing device 10 (FIG. 4). After passing from the annular tip 50 of the hemostasis flushing device 10, the elongate medical device 100 extends into the accessory channel 204 of the attached endoscope 200 to reduce bleeding at the target site (FIG. 7). One or more elongate medical devices may be inserted through the access port 60 of the main body 70 to facilitate hemostasis in a gastrointestinal passageway. For example, a probe and a needle may be inserted into the access port of the hemostasis flushing device and fall within the scope of the present invention.

Referring to FIG. 3, the hemostasis flushing device 10 can include a supporting seal 80 disposed around the lumen 70 of the main body 20. In the embodiment illustrated, the seal 80 is a multi-part or composite seal comprising a proximal seal 84 and a distal seal 82. Both the proximal seal 82 and distal seal 84 are each configured to allow probes, needles, or similar elongate medical devices extending through the lumen 70 of the hemostasis flushing device 10 to pass through while maintaining an adequate seal there about. In other words, each of these seals limits the escape of fluids that may be present within the working channel of the endoscope without inhibiting the insertion or movement of the elongate medical device 100.

In the embodiment illustrated, proximal seal 82 comprises a foam disk having an opening disposed therethrough or some other suitable device for sealing about the elongate medical device 100. The distal seal 84 of the support seal 80 comprises a pair of sealing lips 83, 86, such as a duck bill valve, that are forced in an open position by the elongate medical device 100 as it passes through the access port 60 into the lumen 70 (FIG. 3). When the elongate medical device 100 is removed from the access port 60, the sealing lips 83, 86 are maintained in a closed position wherein any internal reverse flow of blood is prevented from flowing out through the access port 60 (FIG. 4). In the closed position, the access port 60 allows any fluid accumulated in the lumen 70 of the hemostasis flushing device 10 to be removed or flushed from around the elongate medical device 100 by utilizing the flush port 30. The supporting seal 80 also allows the pressure in the lumen 70 to remain constant during medical procedure involving the elongate medical device 100. This configuration can be of particular benefit in preventing bodily fluids such as bile and blood from escaping and contaminating the physician and the working environment.

In the embodiment illustrated, the access port 60 allows the elongate medical device 100 to include a wide range of diameters that are insertable into the access port 60 with ease. Additionally, any blood which normally flows into the lumen 70 is prevented from escaping to the exterior because of the seal 80. The diameter of the access port 60 should be less than the diameter of the longitudinally extending lumen 70 so as to insure that, upon insertion of the elongate medical device 100 into the lumen 70, the seal 80 will not expand against the walls of the main body 20 thereby increasing the difficulty of inserting the elongate medical device 100 and the likelihood of decreasing pressure within the lumen 70.

The design and configuration of these seals, including the types of material from which they are manufactured, are well known to those skilled in the art. An exemplary seal can include slits, torn holes, arranged slits, or penetrable seals. Other seal configurations include duckbill, membrane with a slit (e.g., polystyrene, silicone, or another compliant polymer material), foam seal with small central aperture comprising an open cell or closed cell (e.g., silicon, polyurethane, etc.), or other designs having the ability to seal around the elongate medical device 100 to prevent any proximally migrating fluid from exiting the hemostasis flushing device 10. In another embodiment of the present invention, the hemostasis flushing device 10 may include a tapered lumen 70 having a large diameter proximal end 24 and a small diameter distal end 22 to facilitate the flow of blood from the proximal end 24 to the distal end 22 of the hemostasis flushing device 10 during flushing and to reduce the level of overflow fluid passing through the ports 30, 60.

The flush port 30 comprises a proximal end 34 and a distal end 32. The distal end 32 of the flush port 30 is engaged to the main body 20 of the hemostasis flushing device 10. The proximal end 34 of the flush port 30 extends outwardly from the main body 20 of the hemostasis flushing device 10. The flush port 30 provides a passageway for a liquid, such as flushing fluid, to enter the lumen 70 of the main body 20 and pass to the target site of an active bleed. Preferably, the flush port 30 facilitates attachment of tubing or the like to permit insertion or withdrawal of fluids from the lumen 70 during use.

The flush port 30 can operate as an inlet port or an outlet port. As an inlet port, the flush port 30 operates as a pathway for passing a flushing fluid into the accessory channel 204 of the endoscope 200, and in particular, into the cavity between the outside of the elongate medical device 100 and the inside of the accessory channel 204. As an outlet port, the flush port 30 operates as a pathway for removing fluid from the accessory channel 204 of the endoscope 200 and the lumen 20 of the main body 20. Additionally, the flush port 30 can be used to prevent fluid from accumulating within the hemostasis flushing device 10 during active bleeding at the target site of the gastrointestinal passageway. In particular, the flush port 30 allows blood to be drawn from the site during periods of active bleeding without the removal of any complimentary medical devices.

The flush port 30 allows a medical device, such as a syringe, needle or other infusion device, to be attached to the flush port 30 to transport fluid to the target site. The flush port 30 provides a means of access for delivering flushing fluid to the target site of a patient without having to remove the attached endoscope 200. Thus, endoscopic procedures are easier to perform due to the ability to flush fluid around the elongate medical device 100 as it enters into the accessory channel 204 of the endoscope 200. The flush port 30 further provides increased pressure levels in the main body 20 to maintain pressure during flushing of the target site of the patient. Alternatively, the flush port 30 can be used to deliver medicine to the vascular system of the patient.

In a preferred embodiment, the flush port 30 comprises a seal 36 providing secured access to the main body 20 of the hemostasis flushing device 10. The seal 36 of the flush port 30 is configured and sized to receive an infusion device inserted into the flush port 30 by a variety of methods. The infusion device, such as a needle or syringe, can be inserted directly into the flush port 30 wherein the infusion device is engaged by an interior surface 57 of the seal 36. Upon insertion of the infusion device into the flush port 30, the interior surface 57 of the seal 36 forms a seal with the infusion device providing secured access to the lumen 70 of the main body 20. When the infusion device is removed from the flush port 30, the seal 36 prevents fluid from passing out through the passageway of the flush port 30. The seal 36 comprises a sealing mechanism 57, such as a septum, that forms a liquid tight connection between the flush port 30 and the lumen 70 to prevent air and fluid from escaping from the lumen 70 of the hemostasis flushing device 10.

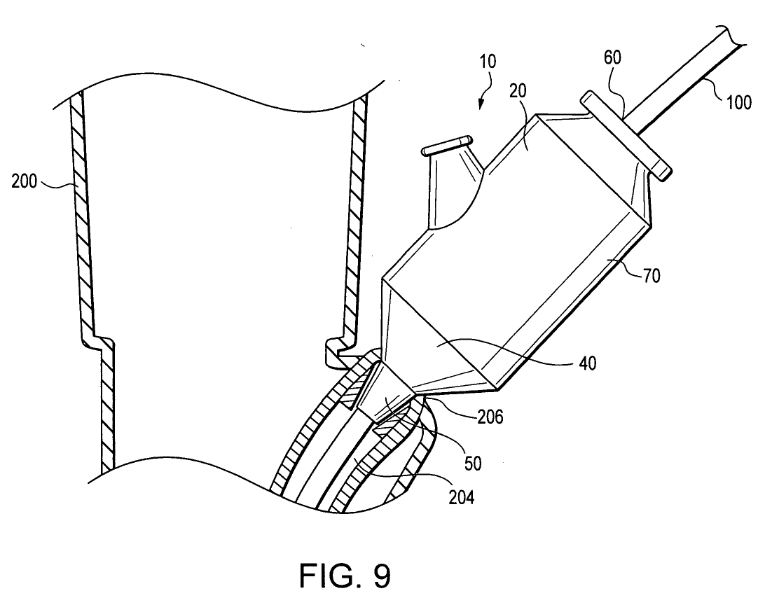

The attachment port 40 has a proximal end 44 and a distal end 42. The proximal end 44 of the attachment port 40 is engaged to the main body 20 while the distal end 42 of the attachment port 40 is engaged to the annular tip 50. The annular tip 50 comprises a proximal end 54 and a distal end 52. As shown in FIG. 1, the proximal end 54 of the annular tip 50 is engaged to the distal end 42 of the attachment port 40. The attachment port 40 is frictionally engaged to the opening of the accessory channel 204 of the endoscope 200, wherein the annular tip 50 is received and secured in opening of the accessory channel 204 of the endoscope 200 (see FIG. 9). The annular tip 50 and the attachment port 40 create a seal with the accessory channel 204 of the endoscope 200 and allows the inserted elongate medical device 100 to extend through the lumen 70 of the hemostasis flushing device 10 into the accessory channel 204 of the endoscope 200. Additionally, the annular tip 50 provides a passageway for fluid as it travels directly from the flush port 30 into the accessory channel 204 of the endoscope 200 for flushing along the accessory channel 204 at the target site. The annular tip 50 also provides a lumen for receiving the inserted elongate medical device 100 as the device 100 extends from the lumen 70 and into the endoscope 200. The attachment port 40 may be engaged to the accessory channel of any suitable endoscope utilizing frictional engagement, locking engagement or the like and fall within the scope of the present invention.

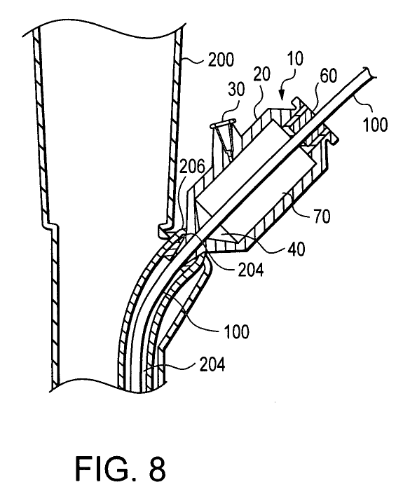

FIG. 6 illustrates an exemplary endoscope 200 and a metal insert 202 leading into the accessory channel 204 of the endoscope 200. The accessory channel 204 provides access to a working channel (not shown) that extends distally through the interior of the endoscope 200. The metal insert 202 has a lip 206 and may be covered by an access port cover (not shown), which may be removed to access the metal insert 202 and the accessory channel 204. FIG. 7 illustrates the hemostasis flushing device 10 frictionally engaged or otherwise attached to the endoscope 200. In particular, the attachment port 40 of the hemostasis flushing device 10 is attached or removed from the lip 206 of the endoscope 200 via frictional fit or the like. The annular tip 50 extends into the accessory channel 204 of the endoscope 200 as the attachment port 40 engages the lip 206 of the endoscope 200 (FIG. 8-9).

An exemplary embodiment of an elongate medical device 100, such as an injection needle (not shown) is insertable into the access port 60 of the hemostasis flushing device 10. The injection needle is used for the injection of fluids or medicine into the gastrointestinal mucosa. The injection needle can be used in conjunction with the hemostasis flushing device 10 to flush the target injection area if desired. Additionally, the injection needle can be navigated with another elongate medical device, such as an inner injection catheter, through a gastrointestinal passageway. The injection needle is navigated through a gastrointestinal passageway toward a point of treatment. Once positioned within the vessel, a second elongate medical device, the inner injection catheter, can be placed over the injection needle and moved along its length toward the point of treatment.

In another exemplary embodiment of an elongate medical device 100, an argon plasma coagulator (not shown) is insertable into the access port 60 of the hemostasis flushing device 10. The argon plasma coagulator is used for open endoscopic applications in conjunction with the hemostasis flushing device 10. The argon plasma coagulator conducts monopolar electrosurgical current to tissue via an ionized argon gas stream (argon plasma) for hemostasis of large surface bleeding. Various probe devices that are utilized with the argon plasma coagulator are insertable into the access port 60 of the hemostasis flushing device 10 and extended along the lumen 70 into the accessory channel 204 of the endoscope 200. The probes can be introduced through the opening of the access port 60 and passed through the lumen 70 of the main body 20. After extending through the lumen 70, the probes pass through the opening of the annular tip 50 and into the accessory channel 204 of the attached endoscope 200. The probes can include diameters of varying sizes. The access port 60 of the hemostasis flushing device 10 can accommodate the varying sizes of the probes while allowing the probes to inserted or removed with ease.

The elongate medical device 100 may be treated with a hydrophilic coating or hybrid polymer mixture, such as those based on polyvinyl puroladine and cellulose esters in organic solvent solutions. These solutions make the medical devices particularly lubricous when in contact with body fluids, which aids in navigation. The coating may be applied by dipping, molding, or spraying a suitable material, such as polytetraflouroethylene (PTFE), urethane, and/or other polymeric coatings, directly to the elongate medical device 100.

In another embodiment of the present invention, a sheath and dilator (not shown) may be inserted into the access port 60 of the hemostasis flushing device 10. The dilator can be received by a sheath such that the sheath encloses the dilator, which can be inserted through the access port 60 and passed through the lumen 70 and exited through the annular tip 50. After exiting the annular tip 50, the sheath and dilator can be advanced through the endoscope 200. The hemostasis flushing device 10 also prevents any blood flowing between the attached devices from escaping out of the flush port 30 or access port 60 as the devices, such as a sheath and dilator, are extended through the lumen 70 of the hemostasis flushing device 10.

Novel features of the disclosed hemostasis flushing device 10 can be successfully used in a variety of medical procedures. In particular, the disclosed hemostasis flushing device 10 can be used in medical procedures in which one or more elongate hemostasis instruments such as a needle or probe needs to be inserted into the endoscope 200 or other medical instrument.

The above figures and disclosures are intended to be illustrative and not exhaustive. This description will suggest many variations and alternatives to one of ordinary skill in the art. All such variations and alternatives are intended to be encompassed within the scope of the attached claims. Those familiar with the are may recognized other equivalents to the specific embodiments described herein which equivalents are also intended to encompass by the attached claims.

1. A hemostasis flushing device, comprising:
a main body having a proximal end and a distal end wherein a lumen extends between the proximal end and the distal end of the main body for inserting an elongate medical device therethrough;
a flush port disposed between the proximal end and the distal end of the main body for passing fluid into or out from the lumen of the main body;
an access port disposed along the proximal end of the main body for receiving the elongate medical device extending through the lumen of the main body; and
an attachment port disposed along the distal end of the main body wherein the attachment port is connectable to an endoscope, the attachment port being configured for receiving the medical device extending through the lumen of the main body.

2. The hemostasis flushing device of claim 1 wherein the attachment port comprises
an annular tip for receiving the elongate medical device extending through the lumen of the main body, the annular tip being configured for insertion into an access port of an endoscope.

3. The hemostasis flushing device of claim 1 further comprising an infusion device attached to the flush port for flushing fluid through the flush port.

4. The hemostasis flushing device of claim 1 wherein the flush port is in fluid communication with the lumen of the main body, the flush port being configured to permit ingress and egress of fluid from the lumen of the hemostasis flushing device.

5. The hemostasis flushing device of claim 1 wherein the flush port includes a seal disposed along an interior surface of the flush port.

6. The hemostasis flushing device of claim 1, wherein the endoscope includes a metal insert configured for frictional engagement to the attachment port of the hemostasis flushing device.

7. The hemostasis flushing device of claim 1 wherein the endoscope includes a metal insert configured for locking engagement to the attachment port of the hemostasis flushing device.

8. The hemostasis flushing device of claim 1 wherein the access port includes a seal disposed along an interior surface of the access port.

9. The hemostasis flushing device of claim 1 wherein the proximal end of the main body includes a larger diameter and the distal end of the main body includes a smaller diameter for facilitating fluid flow within the lumen of the hemostasis flushing device.

10. A hemostasis flushing device, comprising:
a main body having a proximal end and a distal end wherein a lumen extends between the proximal end and the distal end of the main body for inserting an elongate medical device therethrough;
a flush port disposed between the proximal end and the distal end of the main body for passing fluid into or out from the lumen of the main body, wherein the flush port comprises a seal disposed along an interior surface of the flush port;
an access port disposed along the proximal end of the main body for receiving the elongate medical device extending through the lumen of the main body, wherein the access port comprises a seal disposed along an interior surface of the access port; and
an attachment port disposed along the distal end of the main body wherein the attachment port is connectable to an endoscope, the attachment port being configured for receiving the elongate medical device extending through the lumen of the main body.

11. The hemostasis flushing device of claim 10 wherein the attachment port comprises an annular tip for receiving the elongate medical device extending through the lumen of the main body, the annular tip being configured for insertion into an access port of an endoscope.

12. The hemostasis flushing device of claim 10 further comprising an infusion device
attached to the flush port for flushing fluid through the flush port.

13. The hemostasis flushing device of claim 10 wherein the flush port is in fluid communication with the lumen of the main body, the flush port being configured to permit ingress and egress of fluid from the lumen of the hemostasis flushing device.

14. The hemostasis flushing device of claim 10, wherein the endoscope includes a metal insert configured for frictional engagement to the attachment port of the hemostasis flushing device.

15. The hemostasis flushing device of claim 1 wherein the endoscope includes a metal insert configured for locking engagement to the attachment port of the hemostasis flushing device.

16. The hemostasis flushing device of claim 10 wherein the seal of the flush port is selected from the group consisting of a duck bill, membrane and foam.

17. The hemostasis flushing device of claim 10 wherein the seal of the access port is selected from the group consisting of a duck bill, membrane and foam.

18. The hemostasis flushing device of claim 10 wherein a plurality of elongate medical devices are inserted through the lumen of the main body for facilitating hemostasis.

19. The hemostasis flushing device of claim 10 wherein the proximal end of the main body includes a larger diameter and the distal end of the main body includes a smaller diameter for facilitating fluid flow within the lumen of the hemostasis flushing device.

20. A method of flushing around an elongate medical device, the method comprising the steps of:
providing a hemostasis flushing device comprising a main body having a proximal end and a distal end wherein a lumen extends between the proximal end and the distal end of the main body for inserting an elongate medical device, a flush port disposed between the proximal end and the distal end of the main body for passing fluid into the lumen of the main body, an access port disposed along the proximal end of the main body for receiving the elongate medical device extending through the lumen of the main body, and an attachment port disposed along the distal end of the main body wherein the attachment port is connectable to an endoscope for receiving the elongate medical device extending through the lumen of the main body;
attaching an endoscope to the attachment port;
inserting an elongate medical device through the lumen of the main body;
connecting an infusion device to the flush port of the hemostasis flushing device; and
flushing fluid through the flush port into the lumen of the main body to remove bodily fluid from around the elongate medical device.
